(19) United States
(12) Patent Application Publication
(10) Pub. No.: US 2005/0008851 A1
(43) Pub. Date: Jan. 13, 2005

(54) Biosensor

(75) Inventors: Toshihide Ezoe, Minami-ashigara-shi (JP); Toshiaki Kubo, Minami-ashigara-shi (JP); Yoshihisa Tsukada, Minami-ashigara-shi (JP); Junichi Yamanouchi, Minami-ashigara-shi (JP); Hideaki Tanaka, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/779,854

(22) Filed: Feb. 18, 2004

(30) Foreign Application Priority Data
Feb. 18, 2003 (JP) 039659/2003
Mar. 5, 2003 (JP) 058729/2003
Mar. 5, 2003 (JP) 058735/2003
Mar. 20, 2003 (JP) 077781/2003
Mar. 20, 2003 (JP) 077782/2003
Apr. 16, 2003 (JP) 111565/2003

(51) Int. Cl. C12M 1/00; G01N 33/557; B32B 27/00

(52) U.S. Cl. 428/336; 204/403.01; 205/792; 427/58; 428/425.8

(57) ABSTRACT

It is an object of the present invention to provide a detection surface used for a biosensor wherein nonspecific adsorption is repressed. The present invention provides a biosensor comprising a substrate coated with a hydrophobic polymer.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor. Further, the present invention relates to a measurement chip which is used for a surface plasmon resonance measurement device, and a method for analyzing an interaction between biomolecules using the measurement chip.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The OCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

When a specific binding reaction between a physiologically active substance and a test substance is measured, the test substance is not necessarily comprised of a single component. There may also be a case where a test substance is required to be measured in a heterogeneous system such as a cell extract. In such a case, if contaminants such as various proteins or lipids are adsorbed on the detection surface nonspecifically, measurement/detection sensitivity is significantly reduced. The fact that nonspecific adsorption is highly likely to occur on the above detection surface has been problematic. Further, instability of baseline at measurement has also been problematic.

In order to solve such problems, several methods have been studied. For example, a method of immobilizing a hydrophilic hydrogel on a metal surface via a linker, so as to repress physical adsorption, has been used (Japanese Patent No. 2815120, U.S. Pat. No. 5,436,161, and Japanese Patent Laid-Open No. 8-193948). However, nonspecific adsorption has not been sufficiently controlled by this method, and instability of baseline at measurement has also been problematic.

### DISCLOSURE OF THE INVENTION

It is an object of the present invention to solve the above problems of the prior art techniques. This is to say, it is an object of the present invention to provide a detection surface used for a biosensor wherein nonspecific adsorption is repressed. It is another object of the present invention to provide a biosensor wherein baseline at measurement is stabilized.

As a result of intensive studies for achieving the above objects, the present inventors have found that a biosensor wherein nonspecific adsorption is repressed can be provided by coating the surface of a substrate with a hydrophobic polymer. Further, the present inventors have found that a biosensor wherein baseline at measurement is stabilized can be provided by coating the surface of a substrate with a film whose swelling degree in pure water at 25° C. is between 1 and 5 with respect to the film thickness in a dry state. The present invention has been completed by these findings.

Thus, the present invention provides a biosensor comprising a substrate coated with a hydrophobic polymer.

Preferably, the biosensor according to the present invention comprises a metal surface or metal film coated with a hydrophobic polymer.

Preferably, the metal surface or metal film comprises a free-electron metal selected from a group consisting of gold, silver, copper, platinum and aluminum.

Preferably, the coating thickness of the hydrophobic polymer is between 1 angstrom and 5,000 angstroms, and more preferably between 10 angstroms and 2,000 angstroms.

Another aspect of the present invention provides a biosensor comprising a substrate coated with a film whose swelling degree in pure water at 25° C. is between 1 and 5 with respect to the film thickness in a dry state.

Preferably, the film whose swelling degree in pure water at 25° C. is between 1 and 5 with respect to the film thickness in a dry state is an organic substance.

Preferably, the film whose swelling degree in pure water at 25° C. is between 1 and 5 with respect to the film thickness in a dry state comprises a high polymer comprising 50% by weight or more of monomers having a solubility in water of 20% by weight or less.

Preferably, the film whose swelling degree in pure water at 25° C. is between 1 and 5 with respect to the film thickness in a dry state comprises a hardening agent.

Preferably, the biosensor comprises a metal surface or metal film coated with a film whose swelling degree in pure water at 25° C. is between 1 and 5 with respect to the film thickness in a dry state.

Preferably, the metal surface or metal film comprises a free-electron metal selected from a group consisting of gold, silver, copper, platinum and aluminum.

Preferably, the biosensor according to the present invention has a functional group capable of immobilizing a physiologically active substance on the outermost surface of the substrate.

Preferably, the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group.

Preferably, the biosensor according to the present invention comprises a substrate coated with a hydrophobic polymer, and wherein a functional group capable of immobilizing a physiologically active substance by covalent bond is introduced in a hydrophobic polymer by chemical treatment of the surface of said substrate.

Preferably, the biosensor according to the present invention comprises a linker for immobilizing a physiologically active substance on a surface of the biosensor.

Preferably, the linker is a linker for immobilizing a physiologically active substance on a surface of the biosensor by chemical bonding.

Preferably, the linker is a linker for immobilizing a physiologically active substance on a surface of the biosensor by covalent bonding.

Preferably, the linker is a compound represented by the formula (1)
X-L-Y . . . formula (1)
wherein X represents a group capable of reacting with a functional group of a hydrophobic polymer, L represents a bivalent linking group, and Y represents a group capable of immobilizing a physiologically active substance.

Preferably, the total number of atoms of L of the formula (1) is 2 to 1000.

Preferably, the biosensor according to the present invention is used in non-electrochemical detection, and more preferably it is used in surface plasmon resonance analysis.

Still another aspect of the present invention provides a method for producing the biosensor according to the present invention, which comprises a step of coating a substrate with a hydrophobic polymer.

Preferably, the method for producing the biosensor further comprises a step of performing chemical treatment of a surface of the substrate.

Preferably, the method for producing the biosensor further comprises a step of reacting the substrate with a hydrophobic polymer with a linker.

Still another aspect of the present invention provides the biosensor according to the present invention wherein a physiologically active substance is bound to the surface by covalent bonding.

Still another aspect of the present invention provides a method for immobilizing a physiologically active substance to the biosensor according to the present invention, which comprises a step of making said biosensor come into contact with said physiologically active substance, so that said physiologically active substance is bound to the surface of said biosensor by covalent bonding.

Still another aspect of the present invention provides a method for detecting or measuring a substance interacting with a physiologically active substance, which comprises a step of making the biosensor according to the present invention, to the surface of which said physiologically active substance is bound by covalent bonding, come into contact with a test substance.

Preferably, a substance interacting with the physiologically active substance is detected or measured by a non-electrochemical method.

Preferably, a substance interacting with the physiologically active substance is detected or measured by surface plasmon resonance analysis.

Still another aspect of the present invention provides a method for detecting or measuring a substance interacting with a physiologically active substance which is bound to the surface of a biosensor comprising a substrate coated with a hydrophobic polymer, wherein the above detection or measurement is carried out in the presence of a surfactant.

Preferably, the surfactant is a nonionic surfactant.

Preferably, a solution containing at least a test substance and a surfactant is allowed to come into contact with a biosensor comprising a substrate coated with hydrophobic polymer, on the surface of which a physiologically active substance is bound by covalent bonding.

Preferably, the concentration of a surfactant contained in the solution containing the test substance and the surfactant is between 0.0001% by weight and 1% by weight.

Still another aspect of the present invention provides a measurement chip used for a surface plasmon resonance measurement device comprising: a dielectric block; a metal film formed on a face of the dielectric block; a light source for generating a light beam; an optical system for allowing said light beam to enter said dielectric block such that total reflection conditions can be obtained at the interface between said dielectric block and said metal film and that components at various incident angles can be contained; and a light-detecting means for detecting the state of surface plasmon resonance by measuring the intensity of the light beam totally reflected at said interface,
said measurement chip being comprised of said dielectric block and said metal film, wherein said dielectric block is formed as one block comprising the entirety of the entrance face and exit face of said light beam and a face on which said metal film is formed, said metal film is integrated with the dielectric block, and said metal film is coated with a hydrophobic polymer.

Preferably, the measurement chip according to the present invention has a functional group capable of immobilizing a physiologically active substance on the surface of the metal film coated with a hydrophobic polymer.

Preferably, the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group.

Preferably, the physiologically active substance is bound to the surface by covalent bonding.

Still another aspect of the present invention provides a method for immobilizing a physiologically active substance to a measurement chip, which comprises a step of allowing the measurement chip according to the present invention to come into contact with the physiologically active substance, so as to bind the physiologically active substance to the surface of the measurement chip by covalent bonding.

Still another aspect of the present invention provides a method for detecting or measuring a substance interacting with a physiologically active substance, which comprises a step of allowing the measurement chip according to the present invention, on the surface of which the physiologically active substance is bound by covalent bonding, to come into contact with a test substance.

Still another aspect of the present invention provides a surface plasmon resonance measurement device having the measurement chip according to the present invention.

### BRIEF DESCRIPTION OF THE DRAWINGS

FIG. 1 shows the plasmon resonance measurement device used in the present invention.

FIG. 2 shows the dielectric block used in the present invention.

### BEST MODE FOR CARRYING OUT THE INVENTION

Embodiments of the present invention will be described below.

The biosensor of the present invention is characterized in that it comprises a substrate coated with a hydrophobic polymer.

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

The hydrophobic polymer used in the present invention is a polymer having no water-absorbing properties. Its solubility in water (at 25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A substrate is coated with a hydrophobic polymer according to common methods. Examples of such a coating method may include spin coating, air knife coating, bar coating, blade coating, slide coating, curtain coating, spray method, evaporation method, cast method, and dip method.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value d was calculated by the following formula. As the cohesive energy (Ecoh) of each functional group and the mol volume (V), those defined by Fedors were used (R. F. Fedors, Polym. Eng. Sci., 14(2), P147, P472(1974)).
Δ=(SEcoh/S V)^{1/2}

The SP values of the hydrophobic polymers and the solvents used in the Examples are shown below;
Solvent: 2-phenoxyethanol: 25.3 against polymethylmethacrylate-polystyrene copolymer (1:1): 21.0
Solvent: acetonitrile: 22.9 against polymethylmethacrylate: 20.3
Solvent: toluene: 18.7 against polystyrene: 21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 1 angstrom and 5,000 angstroms, and particularly preferably between 10 angstroms and 3,000 angstroms.

The biosensor of the present invention may comprises a substrate which is coating with a film whose swelling degree in pure water at 25° C. is between 1 and 5 with respect to the film thickness in a dry state.

In the present invention, the ratio of swelling is expressed by (film thickness in a swelling state)/(film thickness in a dry state). When the ratio of swelling is large, it takes time to stabilize a change in the concentration of salts during measurement, and it thereby causes trouble in microdetection. The ratio of swelling is preferably between 1 and 5, and more preferably between 1 and 2.

Next, a high polymer, which can be used in the present invention and comprises 50% or more monomers by weight with a solubility in water of 20% or less by weight, will be described.

Solubility in water at 25° C. of a monomer, which forms the high polymer used in the present invention, can be measured by the method described in Shin Jikken Kagaku Koza Kihon Sosa 1 (New Experimental Chemistry Course Basic Operations 1) (Maruzen Chemical Co., Ltd., 1975). When the solubility was measured by this method, the solubility in water at 20° C. of the above monomer of the present invention was found to be as follows: the solubility was 0.00% by weight in the case of 2-ethylhexyl methacrylate; it was 0.03% by weight in the case of styrene; it was 1.35% by weight in the case of methyl methacrylate; it was 0.32% by weight in the case of butyl acrylate; and it was 0.03% by weight in the case of butyl methacrylate.

Specific examples of a monomer used in the present invention, whose solubility in water is 20% or less by weight, may include styrene, methyl methacrylate, hexafluoropropane methacrylate, vinyl acetate and acrylonitrile.

In the present invention, a high polymer obtained by copolymerizing the above monomer with solubility in water of 20% or less by weight and a monomer with solubility in water of 20% or more by weight, may also be used.

Specific examples of a monomer with solubility in water of 20% or more by weight may include 2-hydroxyethyl methacrylate, methacrylic acid, acrylic acid and allyl alcohol.

The high polymer of the present invention preferably contains a monomer with solubility in water of 20% or less by weight at a ratio of 50% or more by weight. More preferably, it contains the above monomer with solubility in water of 20% or less by weight at a ratio of 75% or more by weight.

Next, a hardening agent that can be used in the present invention will be described.

In general, hydrophilic polymers including gelatin as a typical example become significantly swollen, when water is added. However, such swelling can be controlled by intramolecular crosslinking. In the present invention, a compound capable of forming such an intramolecular crosslink in a polymer is called a hardening agent. Examples of an intramolecular crosslinking reaction may include a reaction in which a covalent bond is formed (e.g., a polyvalent vinyl sulfone compound, a polyvalent epoxy compound, a polyvalent melamine compound, etc.), and a reaction in which an ion bond is formed (e.g., Al(III) ion, Pd(III) ion, Cr(III) ion, Ca(III) ion, Pb(II) ion, Mg(II) ion, Ba(II) ion, etc). However, in the present invention, the form of an intramolecular crosslinking reaction is not particularly limited.

Specific examples of the above compound are as follows. Examples of a vinyl sulfone compound are as follows:
VS-1
CH₂═CHSO₂CH₂CONHCH₂CH₂NHCOCH₂SO₂CH═CH₂
VS-2
CH₂═CHSO₂CH₂C(OH)HCH₂SO₂CH═CH₂
VS-3
CH₂═CHSO₂CH₂C(OH)HC(OH)HCH₂SO₂CH═CH₂
VS-4
CH₂═CHSO₂CH₂C(OH)HCH₂C(OH)HCH₂SO₂CH═?H2

Denacol EX521 (manufactured by Nagase Co., Ltd.) is an example of an epoxy compound, and Sumitex Resin M-3 (manufactured by Sumitomo Chemical Co., Ltd.) is an example of a melamine compound.

Moreover, Al₃(SO₄)₂ is an example of an inorganic salt.

A substrate is coated with the above-described high polymer according to common methods. Examples of such a coating method may include spin coating, air knife coating, bar coating, blade coating, slide coating, curtain coating, spray method, evaporation method, cast method, and dip method.

The coating thickness of a film is not particularly limited, but it is preferably between 1 angstrom and 5,000 angstroms, and particularly preferably between 10 angstroms and 3,000 angstroms.

Preferably, the metal surface or metal film of the biosensor of the present invention is coated with a hydrophobic polymer. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 1 angstrom and 5,000 angstroms, and particularly preferably between 10 angstroms and 2,000 angstroms. If the thickness exceeds 5,000 angstroms, the surface plasmon phenomenon of a medium cannot be sufficiently detected. Moreover, when an interstitial layer consisting of chrome or the like is provided, the thickness of the interstitial layer is preferably between 1 angstrom and 100 angstroms.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and having excellent workability are preferably used.

The biosensor of the present invention comprising a substrate coated with a hydrophobic polymer preferably has a functional group capable of immobilizing a physiologically active substance on the outermost surface of the substrate. The term “the outermost surface of the substrate” is used to mean “the surface, which is farthest from the substrate,” and more specifically, it means “the surface of a hydrophobic polymer applied on a substrate, which is farthest from the substrate.”

Examples of a preferred functional group may include —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, and a vinyl group. The number of carbon atoms contained in the lower alkyl group is not particularly limited herein. However, it is generally about C1 to C10, and preferably C1 to C6.

A method of introducing such a functional group is as follows:
(1) The surface of a sample coated with a high polymer containing a precursor of a desired functional group is allowed to come into contact with a solution containing chemical species capable of changing the precursor into the desired functional group. For example, an NaOH aqueous solution is allowed to come into contact with the surface of a sample coated with a high polymer containing a —COOCH₃ group (e.g., polymethyl methacrylate), so that a —COOH group is generated on the surface.
(2) The surface of a sample coated with an organic high polymer is oxidized by methods such as ozonation or a plasma treatment. For example, a sample coated with polystyrene is ozonized, so that a group such as —OH, —COOH or —CHO is generated on the surface.

In order to introduce these functional groups into the outermost surface, a method is applied that involves applying a hydrophobic polymer containing a precursor of such a functional group on a metal surface or metal film, and then generating the functional group from the precursor located on the outermost surface by chemical treatment. For example, polymethyl methacrylate, a hydrophobic polymer containing —COOCH₃ group, is applied on a metal film, and then the surface comes into contact with an NaOH aqueous solution (IN) at 40° C. for 16 hours, so that a —COOH group is generated on the outermost surface.

The biosensor of the present invention comprises a substrate coated with a hydrophobic polymer, and may have a linker for immobilizing a physiologically active substance on the surface of the biosensor.

A linker used in the present invention will be explained.

The linker of the present invention means a linker capable of indirectly immobilizing a physiologically active substance and a hydrophobic polymer. Examples of an immobilization method may include a method using an electrostatic interaction, a method using a hydrophobic interaction, and a method using chemical bonds. Among these, a method using chemical bonds is preferably used. Examples of such chemical bonds may include covalent bonds, ion bonds, coordinate bonds and hydrogen bonds. Of these, covalent bonds are most preferably used.

A compound represented by the following formula (1) is a specific example of the linker used in the present invention:
X-L-Y formula (1)
wherein X represents a group capable of reacting with a functional group of a hydrophobic polymer, L represents a bivalent linking group, and Y represents a group capable of immobilizing a physiologically active substance.

In the above formula (1), X represents a group capable of reacting with a functional group of a hydrophobic polymer, and it is preferably one selected from a group consisting of a halogen atom, an amino group, an amino group protected with a protecting group, a carboxyl group, a carboxyl group having a leaving group, a hydroxyl group, a hydroxyl group protected with a protecting group, an aldehyde group, —NHNH₂, —N═C═O, —N═C═S, an epoxy group, and a vinyl group.

A protecting group is used herein to mean a group capable of forming a functional group by deprotecting the above group in a reaction system. For example, protecting groups of an amino group may include a tert-butyloxycarbonyl group (Boc), a 9-fluorenylmethyloxycarbonyl group (Fmoc), a nitrophenylsulfenyl group (Nps), and a dithiasuccinyl group (Dts).

An acyl group is an example of a protecting group of a hydroxyl group.

Examples of a leaving group used herein may include a halogen atom, an alkoxy group, an aryloxy group, an alkylcarbonyloxy group, an arylcarbonyloxy group, a halogenated alkylcarbonyloxy group, an alkylsulfonyloxy group, a halogenated alkylsulfonyloxy group, and arylsulfonyloxy group.

In addition, an ester group generated by combining carboxylic acid, a known dehydrating condensing reagent (e.g., carbodiimides) and an N-hydroxy compound is preferably used as a leaving group.

In the formula (1), L represents a bivalent linking group. The total number of atoms of L is preferably 2 to 1000. Moreover, L is preferably one selected from a group consisting of a substituted or unsubstituted alkyl group, a substituted or unsubstituted alkyleneoxy group, a substituted or unsubstituted aryleneoxy group, and a bivalent binding group in which X in the formula (1) is bound to Y in another molecule, so that the structure is connected to another structure.

In the formula (1), Y represents a group capable of immobilizing a physiologically active substance, and it is preferably one selected from a group consisting of a halogen atom, an amino group, an amino group protected with a protecting group, a carboxyl group, a carboxyl group having a leaving group, a hydroxyl group, a hydroxyl group protected with a protecting group, an aldehyde group, —NHNH₂, —N═C═O, —N═C═S, an epoxy group, and a vinyl group.

The same above groups can be used herein as protecting groups and leaving groups.

Specific examples of a compound represented by the formula (1) are given below. However, compounds represented by the formula (1), which can be used in the present invention, are not limited thereto.

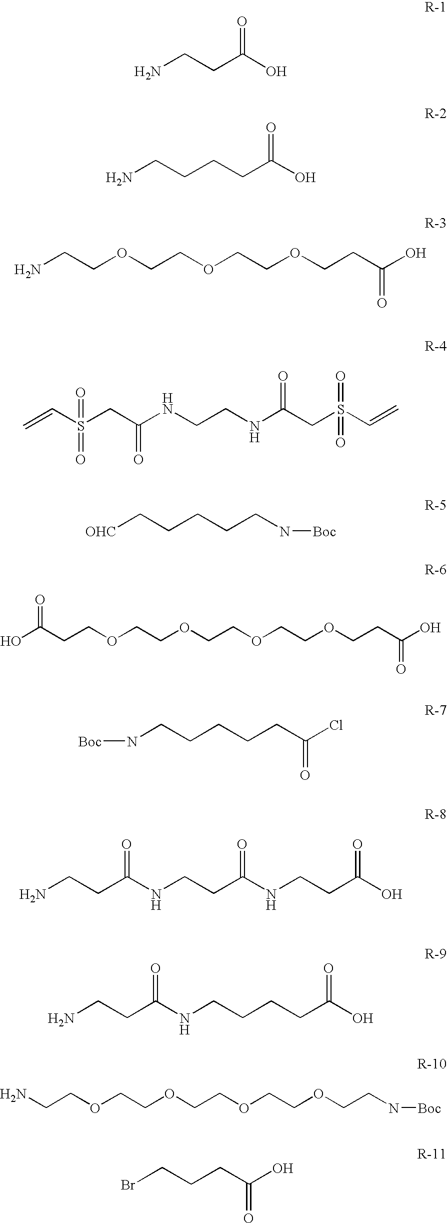

A physiologically active substance is covalently bound to the above-obtained surface used for a biosensor via the above functional group, so that the physiologically active substance can be immobilized on the metal surface or metal film.

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against 0 antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme, or nucleic acid, an amino group, thiol group or the like of the physiologically active substance is covalently bound to a functional group located on a metal surface, so that the physiologically active substance can be immobilized on the metal surface.

A biosensor to which a physiologically active substance is immobilized as described above can be used to detect and/or measure a substance which interacts with the physiologically active substance.

Thus, the present invention provides a method of detecting and/or measuring a substance interacting with the physiologically active substance immobilized to the biosensor of the present invention, to which a physiologically active substance is immobilized, wherein the biosensor is contacted with a test substance

As such a test substance, for example, a sample containing the above substance interacting with the physiologically active substance can be used.

In the present invention, in the method for detecting or measuring a substance interacting with a physiologically active substance binding to the surface of a biosensor coated with a hydrophobic polymer, the above detection or measurement can be carried out in the presence of a surfactant.

The surfactant used in the present invention is, for example, a nonionic, anionic or cationic surfactant. When the surfactant used in the present invention is an anionic or cationic surfactant, there may be a case where nonspecific adsorption of contaminants reversibly charged with the surfactant is promoted. Accordingly, a nonionic surfactant is preferably used as the surfactant used in the present invention.

Specific examples of such a surfactant may include polyoxyethylene lauryl ether, polyoxyethylene stearyl ether, polyoxyethylene sorbitan monolaurate (Tween™ 20), polyoxyethylene sorbitan monooleate (Tween™ 80), polyoxyethylene octylphenyl ether (Triton™ X-100), polyoxyethylene nonylphenyl ether, polyethylene glycol monostearate, polyoxyethylene sorbitan monopalmitate, glycerol monolaurate, glycerol monopalmitate, glycerol monostearate, glycerol monooleate, pentaerythritol monolaurate, sorbitan monopalmitate, sorbitan monobehenate, sorbitan distearate, diglycerol monooleate, triglycerol dioleate, sodium lauryl sulfate, sodium dodecylbenzenesulfonate, sodium butyl naphthalene sulfonate, cetyl trimethyl ammonium chloride, dodecylamine hydrochloride, lauric acid lauryl amide ethyl phosphate, triethyl cetyl ammonium iodide, oleyl amino diethyl amine hydrochloride, and a basic pyridinium salt such as dodecyl pyridinium hydrochloride. Of these, a nonionic surfactant comprising, as a main ingredient, an ester of fatty acid containing 14 to 22 carbon atoms and a polyhydric alcohol such as sorbitan, sorbitol, glycerin, polyglycerin or propylene glycol, or an alkylene oxide adduct thereof, is particularly preferable.

In order to carrying out the detect or the measurement in the presence of a surfactant, for example, a solution containing a test substance and a surfactant can be allowed to come into contact with a biosensor comprising a substrate coated with hydrophobic polymer, on the surface of which a physiologically active substance is bound by covalent bonding. The concentration of a surfactant contained in the solution containing the test substance and the surfactant used herein is not particularly limited, as long as nonspecific adsorption of contaminants contained in the test substance is repressed therein. It is preferably between 0.0001% by weight and 1% by weight, and more preferably between 0.001% by weight and 0.1% by weight.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The measurement chip of the present invention is used for a surface plasmon resonance measurement device with a structure described in the present specification. The inventive measurement chip is characterized in that it comprises a dielectric block and a metal film formed on a face of the dielectric block, in which the dielectric block is formed as one block comprising the entirety of the entrance face and exit face of the light beam and a face on which the above metal film is formed, the above metal film is integrated with the above dielectric block, and the above metal film is coated with a hydrophobic polymer.

Next, a surface plasmon resonance measurement device comprising the measurement chip of the present invention will be described below.

The surface plasmon resonance measurement device is a device for analyzing the properties of a substance to be measured using a phenomenon whereby a surface plasmon is excited with a lightwave.

The surface plasmon resonance measurement device used in the present invention comprises a dielectric block, a metal film formed on a face of the dielectric block, a light source for generating a light beam, an optical system for allowing the above light beam to enter the above dielectric block such that total reflection conditions can be obtained at the interface between the above dielectric block and the above metal film and that components at various incident angles can be contained, and a light-detecting means for detecting the state of surface plasmon resonance by measuring the intensity of the light beam totally reflected at the above interface.

Moreover, as stated above, the above dielectric block is formed as one block comprising the entity of the entrance face and exit face of the above light beam and a face on which the above metal film is formed, and the above metal film is integrated with this dielectric block. Furthermore, the above metal film is coated with a hydrophobic polymer.

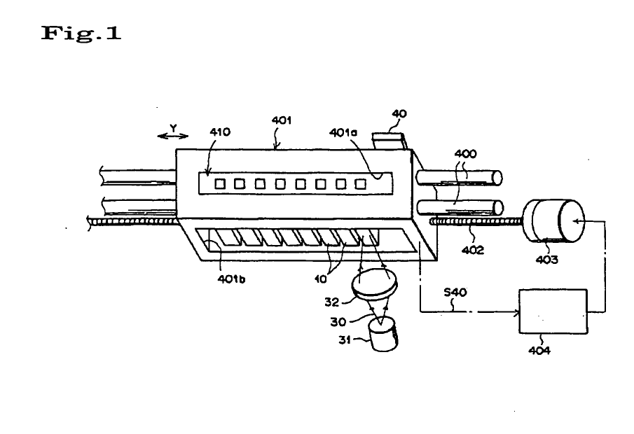

In the present invention, more specifically, a surface plasmon resonance measurement device shown in FIGS. 1 to 32 of Japanese Patent Laid-Open No. 2001-330560, and a surface plasmon resonance device shown in FIGS. 1 to 15 of Japanese Patent Laid-Open No. 2002-296177, can be preferably used. All of the contents as disclosed in Japanese Patent Laid-Open Nos. 2001-330560 and 2002-296177 cited in the present specification are incorporated herein by reference as a part of the disclosure of this specification.

For example, the surface plasmon resonance measurement device described in Japanese Patent Laid-Open No. 2001-330560 is characterized in that it comprises: a dielectric block; a thin metal film formed on a face of the dielectric block; multiple measurement units comprising a sample-retaining mechanism for retaining a sample on the surface of the thin film; a supporting medium for supporting the multiple measurement units; a light source for generating a light beam; an optical system for allowing the above light beam to enter the dielectric block at various angles so that total reflection conditions can be obtained at the interface between the dielectric block and the metal film; a light-detecting means for measuring the intensity of the light beam totally reflected at the above interface and detecting the state of attenuated total reflection caused by surface plasmon resonance; and a driving means for making the above supporting medium, the above optical system and the above light-detecting means move relative to one another, and successively placing each of the above multiple measurement units in a certain position appropriate to the above optical system and the above light-detecting means, so that the above total reflection conditions and various incident angles can be obtained with respect to each dielectric block of the above multiple measurement units.

It is to be noted that in the above measurement device, the above optical system and light-detecting means are kept in a resting state and the above driving means makes the above supporting medium move.

In such a case, the above supporting medium is desirably a turntable for supporting the above multiple measurement units on a circle centered on a rotation axis, and the above driving means is desirably a means for intermittently rotating this turntable. In this case, a medium for supporting the above multiple measurement units that are linearly arranged in a line may be used as the above supporting medium, and a means that makes such a supporting medium move linearly in an intermittent fashion in the direction in which the above multiple measurement units are arranged may be applied as the above driving means.

Otherwise, on the contrary, it may also be possible that the above supporting medium be retained in a resting state and that the above driving means makes the above optical system and light-detecting means move.

In such a case, the above supporting medium is desirably a medium for supporting the above multiple measurement units on a circle, and the above driving means is desirably a means for intermittently rotating the above optical system and light-detecting means along the multiple measurement units supported by the above supporting medium. In this case, a medium for supporting the above multiple measurement units that are linearly arranged in a line may be used as the above supporting medium, and a means that makes the above optical system and light-detecting means move linearly in an intermittent fashion along the multiple measurement units supported by the above supporting medium may be applied as the above driving means.

Otherwise, when the above driving means has a rolling bearing that supports a rotation axis, the driving means is desirably configured such that after the rotation axis has been rotated to a certain direction and a series of measurements for the above multiple measurement units has been terminated, the above rotation axis is equivalently rotated to the opposite direction, and then it is rotated again to the same above direction for the next series of measurements.

In addition, the above-described measurement device is desirably configured such that the above multiple measurement units are connected in a line with a connecting member so as to constitute a unit connected body and that the above supporting medium supports the unit connected body.

Moreover, in the above-described measurement device, it is desirable to establish a means for automatically feeding a given sample to each sample-retaining mechanism of the multiple measurement units supported by the above supporting medium.

Furthermore, in the above-described measurement device, it is desirable that the dielectric block of the above measurement unit be immobilized to the above supporting medium, that a thin film layer and a sample-retaining mechanism of the measurement unit be unified so as to constitute a measurement chip, and that the measurement chip be formed such that it is exchangeable with respect to the above dielectric block.

When such a measurement chip is applied, it is desirable to establish a cassette for accommodating a multiple number of the measurement chips and a chip-supplying means for successively taking a measurement chip out of the cassette and supplying it in a state in which it is connected to the above dielectric block.

Otherwise, it may also be possible to unify the dielectric block of the measurement unit, the thin film layer and the sample-retaining mechanism, so as to constitute a measurement chip, and it may also be possible for this measurement chip to be formed such that it is exchangeable with respect to the above supporting medium.

When a measurement chip has such a structure, it is desirable to establish a cassette for accommodating a multiple number of measurement chips and a chip-supplying means for successively taking a measurement chip out of the cassette and supplying it in a state in which it is supported by the supporting medium.

The above optical system is desirably configured such that it makes a light beam enter the dielectric block in a state of convergent light or divergent light. Moreover, the above light-detecting means is desirably configured such that it detects the position of a dark line generated due to attenuated total reflection, which exists in the totally reflected light beam.

Furthermore, the above optical system is desirably configured such that it makes a light beam enter the above interface in a defocused state. In this case, the beam diameter of the light beam at the above interface in a direction wherein the above supporting medium moves is desirably ten times or greater the mechanical positioning precision of the above supporting medium.

Still further, the above-described measurement device is desirably configured such that the measurement unit is supported on the upper side of the above supporting medium, such that the above light source is placed so as to project the above light beam from a position above the above supporting medium to downwards, and such that the above optical system comprises a reflecting member for reflecting upwards the above light beam projected to downwards as described above and making it proceed towards the above interface.

Still further, the above-described measurement device is desirably configured such that the above measurement unit is supported on the upper side of the above supporting medium, such that the above optical system is constituted so as to make the above light beam enter the above interface from the downside thereof, and such that the above light-detecting means is placed in a position above the above supporting medium with a light-detecting plane thereof facing downwards, as well as comprising a reflecting member for reflecting upwards the totally reflected light beam at the above interface and making it proceed towards the above light-detecting means.

What is more, the above-described measurement device desirably comprises a temperature-controlling means for maintaining the temperature of the above measurement unit before and/or after being supported by the above supporting medium at a predetermined temperature.

Moreover, the above-described measurement device desirably comprises a means for stirring the sample stored in the sample-retaining mechanism of the measurement unit supported by the above supporting medium before detecting the state of attenuated total reflection as mentioned above.

Furthermore, in the above-described measurement device, it is desirable to establish in at least one of the multiple measurement units supported by the above supporting medium a standard solution-supplying means for supplying a standard solution having optical properties associated with the optical properties of the above sample, as well as a correcting means for correcting data regarding the above attenuated total reflection state of the sample based on the data regarding the above attenuated total reflection state of the above standard solution.

In such a case, if the sample is obtained by dissolving a test substance in a solvent, it is desirable that the above standard solution-supplying means be a means for supplying the above solvent as a standard solution.

Still further, the above measurement device desirably comprises: a mark for indicating individual recognition information; a reading means for reading the above mark from the measurement unit used in measurement; an inputting means for inputting sample information regarding the sample supplied to the measurement unit; a displaying means for displaying measurement results; and a controlling means connected to the above displaying means, inputting means and reading means, which stores the above individual recognition information and sample information of each measurement unit while associating them with each other, as well as making the above displaying means display the measurement results of the sample retained in a certain measurement unit while associating them with the above individual recognition information and sample information of each measurement unit.

When a substance interacting with a physiologically active substance is detected or measured using the above-described measurement device, a state of attenuated total reflection is detected in a sample contained in one of the above measurement units, and thereafter, the above supporting medium, optical system and light-detecting means are moved relative to one another, so that a state of attenuated total reflection is detected in a sample contained in another measurement unit. Thereafter, the above supporting medium, optical system and light-detecting means are again moved relative to one another, so that a state of attenuated total reflection is detected again the sample contained in the above one measurement unit, thereby completing the measurement.

The present invention will be further specifically described in the following examples. However, the examples are not intended to limit the scope of the present invention.

### EXAMPLES

### Example A-1

### Production of Chip for Biosensor

(1) Production of Chip for Biosensor Coated with Polymethyl Methacrylate

A cover glass with a size of 1 cm×1 cm, onto which gold had been evaporated such that the thickness of a gold film became 500 angstroms, was treated with a Model-208 UV-ozone cleaning system (TECHNOVISION INC.) for 30 minutes. Thereafter, the cover glass was placed in a spin coating machine (MODEL ASS-303, manufactured by ABLE), and it was then rotated at 1,000 rpm. 50 μl of a methyl ethyl ketone solution containing polymethyl methacrylate (2 mg/ml) was added dropwise to the center of the gold-evaporated cover glass. After 2 minutes, the rotation was terminated. Film thickness was measured by ellipsometry (In-Situ Ellipsometer MAUS-101, manufactured by Five Lab). As a result, the thickness of the polymethyl methacrylate film was found to be 200 angstroms. This sample is called a PMMA surface chip.

(2) Introduction of COOH Group onto the PMMA Surface

The above produced cover glass coated with polymethyl methacrylate was immersed in an NaOH aqueous solution (1N) at 40° C. for 16 hours, and it was then washed with water 3 times. This sample is called a PMMA/COOH surface chip.

### Comparative Example A-1

### Production of Gold Surface Chip Without Surface Coating

A cover glass with a size of 1 cm×1 cm, onto which gold had been evaporated such that the thickness of a gold film became 500 angstroms, was treated with a Model-208 UV-ozone cleaning system (TECHNOVISION INC.) for 30 minutes. This sample is called a gold surface chip.

### Comparative Example A-2

### Production of Chip for Biosensor Coated with SAM Compound (7-carboxy-1-heptanethiol)(SAM: Self-Assembled Membrane)

A cover glass with a size of 1 cm×1 cm, having a gold-evaporated film of a thickness of 50 nm, was treated with an ozone cleaner for 30 minutes. Thereafter, it was immersed in an ethanol solution containing 1 mM 7-carboxy-1-heptanethiol (Dojin Chemicals) so as to carry out surface treatment at 25° C. for 18 hours. Thereafter, the glass was washed at 40° C. with ethanol 5 times, then with a mixed solvent of ethanol/water once, and then with water 5 times. This sample is called an SAM surface chip.

### Example A-2

### Evaluation of Performance of Chip for Biosensor

(1) Measurement of Nonspecific Adsorption of Proteins

Since nonspecific adsorption of proteins on the surface of a biosensor causes noise, such adsorption is preferably as low as possible. Using the following samples 1-1 to 1-4, nonspecific adsorption of BSA (manufactured by Sigma) and avidin (manufactured by Nacalai Tesque) was examined.
Sample 1-1: a gold surface chip that was not subjected to surface treatment (produced by the method in Comparative Example A-1)
Sample 1-2: a chip obtained by blocking the COOH group of the SAM surface chip (produced by the method in Comparative Example A-2) with ethanolamine
Sample 1-3: a PMMA surface chip (produced by the method in Example A-1 (1))
Sample 1-4: a chip obtained by blocking the COOH group of the PMMA/COOH surface chip (produced by the method in Example A-1 (2)) with ethanolamine

The COOH group of each of the above samples 1-2 and 1-4 was blocked with ethanolamine by the following method. Each chip was placed on the cartridge block of a commercially available surface plasmon resonance biosensor (BIACORE 3000 manufactured by Biacore K.K.), and 100 μl of a mixed solution of 1-ethyl-2,3-dimethylaminopropylcarbodiimide (400 mM) and N-hydroxysuccinimide (100 mM) was fed to a measuring cell thereof at a flow rate of 10 μl/min. Thereafter, 100 μl of an ethanolamine/HCl solution (1 M, pH 8.5) was fed thereto at a flow rate of 10 μl/min.

Each of the above samples 1-1 to 1-4 was placed on the cartridge block of the surface plasmon resonance biosensor (BIACORE 3000 manufactured by Biacore K.K.), and 50 μl of BSA solution (1 mg/ml, HBS-EP buffer (manufactured by Biacore K.K., pH 7.4)) or avidin solution (1 mg/ml, HBS-EP buffer) was fed to a measuring cell thereof at a flow rate of 10 μl/min. The HBS-EP buffer consisted of 0.01 mol/l (pH 7.4) HEPES (N-2-hydroxyethylpiperazine-N′-2-ethanesulfonic acid), 0.15 mol/l NaCl, 0.003 mol/l EDTA, and 0.005 weight % Surfactant P20. The amount of change of the resonance signal (RU value) at 3 minutes after completion of the addition of BSA solution or avidin solution was defined as a nonspecifically adsorbed amount of each protein.

(2) Measurement of Interaction Between Protein and Test Compound

Neutral avidin (manufactured by PIERCE) was immobilized to the following samples, and an interaction with D-biotin (manufactured by Nacalai Tesque) was measured by the method described below.
Sample 2-1: a SAM surface chip (produced by the method in Comparative Example A-2)
Sample 2-2: a PMMA/COOH surface chip (produced by the method in Example A-1 (2))

Each of the above samples 2-1 and 2-2 that were chips for immobilizing physiologically active substances was placed on the cartridge block of the surface plasmon resonance biosensor (BIACORE 3000 manufactured by Biacore K.K.), and 100 μl of a mixed solution of 1-ethyl-2,3-dimethylaminopropylcarbodiimide (400 mM) and N-hydroxysuccinimide (100 mM) was fed to a measuring cell thereof at a flow rate of 10 μl/min. Thereafter, 300 μl of a neutral avidin solution (100 μg/ml, HBS-N buffer (manufactured by Biacore K.K., pH 7.4)) was poured into a measuring cell thereof at a flow rate of 10 μl/min, so that the neutral avidin was immobilized on the surface of each sample by covalent bonding. The HBS-N buffer consisted of 0.01 mol/l (pH 7.4) HEPES (N-2-hydroxyethylpiperazine-N′-2-ethanesulfonic acid) and 0.15 mol/l NaCl. The amount of change of resonance signal (RU value) obtained before the addition of neutral avidin and at 3 minutes after completion of the addition was defined as the immobilized amount of neutral avidin (RU value).

Thereafter, 100 μl of an ethanolamine/HCl solution (1 M, pH 8.5) was fed to the measuring cell at a flow rate of 10 μl/min, so that COOH groups remaining without reacting with neutral avidin were blocked.

Subsequently, 100 μl of D-biotin (1 μg/ml, HBS-N buffer) was fed to the measuring cell at a flow rate of 10 μl/min. The amount of change of resonance signal (RU value) obtained before the addition of D-biotin and at 3 minutes after completion of the addition was defined as the amount of D-biotin binding to neutral avidin.

(3) Results

Table 1 shows measurement results of the nonspecific adsorption of a protein, and Table 2 shows measurement results of an interaction between a protein and a test compound.

TABLE 1

nonspecific adsorption
(RU value)

| Sample | BSA | avidin | Remarks |
|---|---|---|---|
| 1-1 | 594 | 844 | Comparative |
| 1-2 | 207 | 618 | Comparative |
| 1-3 | 13 | 52 | Invention |
| 1-4 | 28 | 85 | Invention |

TABLE 2

| | Binding amount of | Binding amount of | |
|---|---|---|---|
| Sample | neutral avidin (RU value) | D-biotin (RU value) | Remarks |
| 2-1 | 3020 | 27 | Comparative |
| 2-2 | 2840 | 28 | Invention |

From the results shown in Table 1, it has been found that the present invention provides a surface causing an extremely small degree of nonspecific adsorption of proteins. From the results shown in Table 2, it has been found that the present invention enables immobilization of proteins and detection of a test compound, as in the conventional methods. This is to say, the present invention provides a surface used for a biosensor having an excellent ability of repressing the nonspecific adsorption of proteins.

### Example B-1

### Production of Chip for Biosensor

(1) Production of Chip for Biosensor Coated with Polymethyl Methacrylate

A chip for biosensor coated with polymethyl methacrylate was produced as in Example A-1 (1). This sample is called a PMMA surface chip.

(2) Introduction of COOH Group onto the PMMA Surface

A COOH group was introduced onto the PMMA surface as in Example A-1 (2). This sample is called a PMMA/COOH surface chip.

(3) Production of Surface on which Physiological Active Substance is Immobilized

The above produced PMMA/COOH surface chip was placed on the cartridge block of a commercially available surface plasmon resonance biosensor (BIACORE 3000, manufactured by Biacore K.K.), and 300 μl of a mixed solution of 1-ethyl-2,3-dimethylaminopropylcarbodiimide (400 mM) and N-hydroxysuccinimide (100 mM) was fed to a measuring cell thereof at a flow rate of 10 μl/min.

Thereafter, 300 μl of a 5-aminovaleric acid solution (1 M, pH 8.5) was fed thereto at a flow rate of 10 μl/min. This sample is called a PMMA/val surface chip.

### Comparative Example B-1

### Production of Gold Surface Chip without Surface Coating

A gold surface chip without surface coating was produced as in Comparative Example A-1. This chip is called a gold surface chip.

### Comparative Example B-2

### Production of Chip for Biosensor Coated with SAM Compound (7-carboxy-1-heptanethiol) (SAM: Self-Assembled Membrane)

A chip for biosensor coated with SAM compound was produced as in Comparative Example A-2. This chip is called an SAM surface chip.

### Example B-2

### Evaluation of Performance of Chip for Biosensor

(1) Measurement of Nonspecific Adsorption of Proteins

Since the nonspecific adsorption of proteins on the surface of a biosensor causes noise, such adsorption is preferably as low as possible. Using the following samples 1-1 to 1-4, the nonspecific adsorption of BSA (manufactured by Sigma) and avidin (manufactured by Nacalai Tesque) was examined.
Sample 1-1: a gold surface chip that was not subjected to surface treatment (produced by the method in Comparative Example B-1)
Sample 1-2: a chip obtained by blocking a COOH group of the SAM surface chip (produced by the method in Comparative Example B-2) with ethanolamine
Sample 1-3: a chip obtained by blocking a COOH group of the PMMA/COOH surface chip (produced by the method in Example B-1 (2)) with ethanolamine
Sample 1-4: a chip obtained by blocking a COOH group of the PMMA/val surface chip (produced by the method in Example B-1 (3)) with ethanolamine

The COOH group of each of the above samples 1-2 and 1-4 was blocked with ethanolamine by the following method. Each chip was placed on the cartridge block of a commercially available surface plasmon resonance biosensor (BIACORE 3000 manufactured by Biacore K.K.), and 100 μl of a mixed solution of 1-ethyl-2,3-dimethylaminopropylcarbodiimide (400 mM) and N-hydroxysuccinimide (100 mM) was fed to a measuring cell thereof at a flow rate of 10 μl/min. Thereafter, 100 μl of an ethanolamine/HCl solution (1 M, pH 8.5) was fed to the measuring cell at a flow rate of 10 μl/min.
Buffer A: HBS-N buffer (manufactured by Biacore K.K., pH 7.4)
Buffer B: HBS-EP buffer (manufactured by Biacore K.K., pH 7.4)
Buffer C: 0.005 g of tween 20 (manufactured by Aldridge) was mixed with the HBS-N buffer (manufactured by Biacore, K.K., pH 7.4) so as to prepare a total 100 ml of buffer.
Buffer D: 0.005 g of TRITON-X100 (manufactured by ICI) was mixed with the HBS-N buffer (manufactured by Biacore, K.K., pH 7.4) so as to prepare a total 100 ml of buffer.

The HBS-N buffer used above consisted of 0.01 mol/l (pH 7.4) HEPES (N-2-hydroxyethylpiperazine-N′-2-ethanesulfonic acid) and 0.15 mol/l NaCl. In addition, the HBS-EP buffer consisted of 0.01 mol/l (pH 7.4) HEPES, 0.15 mol/l NaCl, 0.003 mol/l EDTA, and 0.005 weight % surfactant P20.

Each of the above samples 1-1 to 1-4 was placed on the cartridge block of the surface plasmon resonance biosensor (BIACORE 3000 manufactured by Biacore K.K.), and the aforementioned buffers A to D (pH 7.4) were then fed thereto for 10 minutes. Thereafter, 50 μl of BSA solution (1 mg/ml, dissolved with the above buffers A to D (pH 7.4)) or avidin solution (1 mg/ml, dissolved with the above buffers A to D (pH 7.4)) was fed to a measuring cell thereof at a flow rate of 10 μl/min. The amount of change of resonance signals (RU value) measured at 3 minutes after completion of the addition of the BSA or avidin solution was defined as a nonspecifically adsorbed amount of each protein.

(2) Results

Table 3 shows measurement results of the nonspecific adsorption of proteins.

TABLE 3
non specific adsorption (RU value)

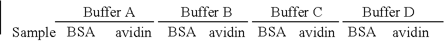

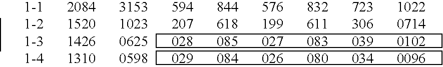

The box portion corresponds to the present invention.

From the results shown in Table 3, it has been found that the present invention provides a detection or measurement method that causes extremely small degree of nonspecific adsorption of proteins.

### Example C-1

### Production of Chip for Biosensor

(1) Production of Chip for Biosensor Coated with Polymethyl Methacrylate

A chip for biosensor coated with polymethyl methacrylate was produced as in Example A-1 (1). This sample is called a PMMA surface chip.

(2) Introduction of COOH Group onto the PMMA Surface

A COOH group was introduced onto the PMMA surface as in Example A-1 (2). This sample is called a PMMA COOH surface chip.

(3) Production of Surface on which Physiological Active Substance is Immobilized

The above produced PMMA/COOH surface chip was placed on the cartridge block of a commercially available surface plasmon resonance biosensor (BIACORE 3000, manufactured by Biacore K.K.), and 300 μl of a mixed solution of 1-ethyl-2,3-dimethylaminopropylcarbodiimide (400 mM) and N-hydroxysuccinimide (100 mM) was fed to a measuring cell thereof at a flow rate of 10 μl/min.

Thereafter, each of 300 μl of an R-1 solution (1M, pH 8.5) and 300 μl of an R-2 solution (1M, pH 8.5) was fed to a measuring cell thereof at a flow rate of 10 μl/min. The chemical structures of the compound R-1 and the compound R-2 are as shown in the above in the present specification. These samples are called a PMMA R-1 surface chip and a PMMA/R-2 surface chip, respectively.

### Comparative Example C-1

### Production of Gold Surface Chip without Surface Coating

A gold surface chip without surface coating was produced as in Comparative Example A-1. This chip is called a gold surface chip.

### Comparative Example C-2

### Production of Chip for Biosensor Coated with SAM Compound (7-carboxy-1-heptanethiol) (SAM: Self-Assembled Membrane)

A chip for biosensor coated with SAM compound was produced as in Comparative Example A-2. This chip is called an SAM surface chip.

### Example C-2

### Evaluation of Performance of Chip for Biosensor

(1) Measurement of Nonspecific Adsorption of Proteins

Since the nonspecific adsorption of proteins on the surface of a biosensor causes noise, such adsorption is preferably as low as possible. Using the following samples 1-1 to 1-5, the nonspecific adsorption of BSA (manufactured by Sigma) and avidin (manufactured by Nacalai Tesque) was examined as in Example A-2 (1).
Sample 1-1: a gold surface chip that was not subjected to surface treatment (produced by the method in Comparative Example C-1)
Sample 1-2: a chip obtained by blocking a COOH group of the SAM surface chip (produced by the method in Comparative Example C-2) with ethanolamine
Sample 1-3: a chip obtained by blocking a COOH group of the PMMA/COOH surface chip (produced by the method in Example C-1(2)) with ethanolamine
Sample 1-4: a chip obtained by blocking a COOH group of the PMMA/R-1 surface chip (produced by the method in Example C-1 (3)) with ethanolamine
Sample 1-5: a chip obtained by blocking a COOH group of the PMMA/R-2 surface chip (produced by the method in Example C-1 (3)) with ethanolamine
   (2) Measurement of Interaction Between Protein and Test Compound

Neutral avidin (manufactured by PIERCE) was immobilized to the following samples, and an interaction with D-biotin (manufactured by Nacalai Tesque) was measured by the same method as in Example A-2 (2).
Sample 2-1: a SAM surface chip (produced by the method in Comparative Example C-2)
Sample 2-2: a PMMA COOH surface chip (produced by the method in Example C-1 (2))
Sample 2-3: a PMMA/R-1 surface chip (produced by the method in Example C-1 (3))
Sample 2-4: a PMMA/R-2 surface chip (produced by the method in Example C-1 (3))
   (3) Results

Table 4 shows measurement results of the nonspecific adsorption of a protein, and Table 5 shows measurement results of an interaction between a protein and a test compound.

TABLE 4

nonspecific
adsorption
(RU value)

| Sample | BSA | avidin | Remarks |
|---|---|---|---|
| 1-1 | 594 | 844 | Comparative |
| 1-2 | 207 | 618 | Comparative |
| 1-3 | 28 | 85 | Comparative |
| 1-4 | 29 | 84 | Invention |
| 1-5 | 27 | 80 | Invention |

TABLE 5

| | Binding amount of | Binding amount of | |
|---|---|---|---|
| Sample | neutral avidin (RU value) | D-biotin (RU value) | Remarks |
| 2-1 | 3020 | 27 | Comparative |
| 2-2 | 2840 | 28 | Comparative |
| 2-3 | 3620 | 40 | Invention |
| 2-4 | 4760 | 52 | Invention |

From the results shown in Table 4, it has been found that the present invention provides a surface causing an extremely small degree of nonspecific adsorption of proteins. From the results shown in Table 5, it has been found that the present invention is more excellent than the conventional methods in immobilization of proteins and detection of a test compound. This is to say, the present invention provides a surface used for a biosensor having an excellent ability of repressing the nonspecific adsorption of proteins.

### Example D-1

### Production of Chip for Biosensor

(1) Comparative Example: Production of Chip for Biosensor Coated with SAM Compound (7-carboxy-1-heptanethiol) (SAM: Self-Assembled Membrane)

A cover glass with a size of 1 cm×1 cm, having a gold-evaporated film of a thickness of 50 nm, was treated with a Model-208 UV-ozone cleaning system (TECHNOVISION INC.) for 30 minutes. Thereafter, it was immersed in an ethanol solution containing 1 mM 7-carboxy-1-heptanethiol (Dojin Chemicals) so as to carry out surface treatment at 25° C. for 18 hours. Thereafter, the glass was washed with ethanol 5 times, then with a mixed solvent of ethanol/water once, and then with water 5 times. This chip is called an SAM treated chip.

(2) Production of Polymethyl Methacrylate (PMMA) Film

A cover glass with a size of 1 cm×1 cm, onto which gold of a thickness of 50 nm had been evaporated, was treated with a Model-208 UV-ozone cleaning system (TECHNOVISION INC.) for 30 minutes. Thereafter, the cover glass was placed in a spin coating machine (MODEL ASS-303, manufactured by ABLE), and it was then rotated at 1,000 rpm. 5 μl of a methyl ethyl ketone solution containing polymethyl methacrylate (4 mg/ml) was added dropwise to the center of the gold-evaporated cover glass. After 2 minutes, the rotation was terminated. The thickness of a polymethyl methacrylate film was measured by ellipsometry (In-Situ Ellipsometer MAUS-101, manufactured by Five Lab). As a result, the thickness of the film was found to be 40 nm.

(3) Production of Chip for Biosensor Comprising Polymethyl Methacrylate Film Treated with NaOH

The sample obtained in (2) above was immersed in an NaOH aqueous solution (1N) at 60° C. for 4 hours, and it was then washed with water 3 times. This sample is called a PMMA/NaOH treated chip.

(4) Production of Polystyrene (PS) Film

A cover glass with a size of 1 cm×1 cm, onto which gold of a thickness of 50 nm had been evaporated, was treated with a Model-208 UV-ozone cleaning system (TECHNOVISION INC.) for 30 minutes. Thereafter, the cover glass was placed in a spin coating machine (MODEL ASS-303, manufactured by ABLE), and it was then rotated at 1,000 rpm. 5 μl of a methyl ethyl ketone solution containing polystyrene (4 mg/ml) was added dropwise to the center of the gold-evaporated cover glass. After 2 minutes, the rotation was terminated. The thickness of a polystyrene film was measured by ellipsometry (In-Situ Ellipsometer MAUS-101, manufactured by Five Lab). As a result, the thickness of the film was found to be 40 nm.

(5) Production of Chip for Biosensor with Polystyrene Film Treated with Ozone

The sample obtained in (4) above was treated with a Model-208 UV-ozone cleaning system (TECHNOVISION INC.) for 30 minutes. This sample is called a PS/ozone treated chip.

### Example D-2

### Evaluation of Performance of Chip for Biosensor

(1) Measurement of Nonspecific Adsorption of Proteins

Since nonspecific adsorption of proteins on the surface of a biosensor causes noise, such adsorption is preferably as low as possible. Nonspecific adsorption of each of BSA (manufactured by Sigma) and avidin (manufactured by Nacalai Tesque) to a chip for a biosensor was examined by the following method.

Each of the SAM treated chip (produced by the method in Example D-1 (1)), the PMMA/NaOH treated chip (produced by the method in Example D-1 (3)) and the PS/ozone treated chip (produced by the method in Example D-1-(5)) was placed on the cartridge block of a commercially available surface plasmon resonance biosensor (BIACORE 3000 manufactured by Biacore K.K.), and 200 μl of a mixed solution consisting of 1-ethyl-2,3-dimethylaminopropylcarbodiimide (400 mM) and N-hydroxysuccinimide (100 mM) was then fed to a measuring cell thereof at a flow rate of 10 μl/min. Thereafter, 100 μl of an ethanol/HCl solution (1 M, pH 8.5) was fed to the measuring cell at a flow rate of 101 l/min.

Subsequently, each of these samples was placed on the cartridge block of the surface plasmon resonance biosensor (BIACORE 3000 manufactured by Biacore K.K.), and 50 μl of BSA solution (2 mg/ml, HBS-EP buffer (manufactured by Biacore K.K., pH 7.4)) or avidin solution (2 mg/ml, HBS-EP buffer) was fed to a measuring cell thereof at a flow rate of 10 μl/min. The amount of change of resonance signals (RU value) measured at 3 minutes after completion of the addition of the BSA or avidin solution was defined as a nonspecifically adsorbed amount of each protein.

The HBS-EP buffer used above consisted of 0.01 mol/l (pH 7.4) HEPES (N-2-hydroxyethylpiperazine-N′-2-ethanesulfonic acid), 0.15 mol/l NaCl, 0.003 mol/l EDTA, and 0.005 weight % surfactant P20.

(2) Measurement of Interaction Between Protein and Test Compound

Neutral avidin (manufactured by PIERCE) was immobilized to each chip for a biosensor, and an interaction with D-biotin (manufactured by Nacalai Tesque) was measured by the method described below.

Each of the SAM treated chip (produced by the method in Example D-1 (1)), the PMMA/NaOH treated chip (produced by the method in Example D-1 (3)) and the PS/ozone treated chip (produced by the method in Example D-1 (5)) was placed on the cartridge block of a commercially available surface plasmon resonance biosensor (BIACORE 3000 manufactured by Biacore K.K.), and 200 μl of a mixed solution of 1-ethyl-2,3-dimethylaminopropylcarbodiimide (400 mM) and N-hydroxysuccinimide (100 mM) was then fed to a measuring cell thereof at a flow rate of 10 μl/min. Thereafter, 300 μl of a neutral avidin solution (100 μg/ml, HBS-N buffer (manufactured by Biacore K.K., pH 7.4)) was poured into the measuring cell at a flow rate of 10 μl/min, so that neutral avidin was immobilized on the surface of each sample by covalent bonding. The amount of change of resonance signals (RU value) measured before the addition of neutral avidin and at 3 minutes after completion of the addition was defined as the immobilized amount of neutral avidin (RU value).

The HBS-N buffer used above consisted of 0.01 mol/l (pH 7.4) HEPES (N-2-hydroxyethylpiperazine-N′-2-ethanesulfonic acid) and 0.15 mol/l NaCl.

Thereafter, 100 μl of an ethanolamine/HCl solution (1 M, pH 8.5) was fed to the measuring cell at a flow rate of 101 l/min, so that COOH groups remaining without reacting with neutral avidin were blocked.

Subsequently, 100 μl of D-biotin (0.5 μg/ml, HBS-N buffer) was fed to the measuring cell at a flow rate of 10 μl/min. The amount of change of resonance signals (RU value) obtained before the addition of D-biotin and at 3 minutes after completion of the addition was defined as the amount of D-biotin binding to neutral avidin.

(3) Results

Table 6 shows measurement results of the nonspecific adsorption of a protein, and Table 7 shows measurement results of an interaction between a protein and a test compound.

TABLE 6

| | | nonspecific | | |
|---|---|---|---|---|
| | | adsorption | | |
| Sample | | (RU value) | | |
| No. | Surface treatment | BSA | avidin | Remarks |
| 1-1 | SAM treated membrane | 321 | 789 | Comparative |
| 1-2 | PMMA/NaOH treated | 20 | 61 | Invention |
| | membrane | | | |
| 1-3 | PS/ozone treated membrane | 45 | 80 | Invention |

TABLE 7

| | | Binding | Binding | |
|---|---|---|---|---|
| | | amount of | amount of | |
| Sample | Surface | neutral avidin | D-biotin | |
| No. | treatment | (RU value) | (RU value) | Remarks |
| 2-1 | SAM treated | 2980 | 21 | Comparative |
| | membrane | | | |
| 2-2 | PMMA/NaOH | 2750 | 23 | Invention |
| | treated membrane | | | |
| 2-3 | PS/ozone treated | 2630 | 21 | Invention |
| | membrane | | | |

From the results shown in Table 6, it has been found that the present invention provides a surface causing an extremely small degree of nonspecific adsorption of proteins. From the results shown in Table 8, it has been found that the present invention enables immobilization of proteins and detection of a test compound as in the conventional methods. This is to say, the present invention provides a surface used for a biosensor having an excellent ability of repressing the nonspecific adsorption of proteins.

The following Examples E-1 and E-2 were carried out using the device of FIG. 22 of Japanese Patent Laid-open No. 2001-330560 (hereinafter referred to as the surface plasmon resonance measurement device of the present invention), and the a dielectric block of FIG. 23 of Japanese Patent Laid-open No. 2001-330560 (hereinafter referred to as the dielectric block of the present invention).

### Example E-1

### Production of Measurement Chip

(1) Comparative Example: Production of Measurement Chip Coated with Dextran

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5.0 mM 11-hydroxy-1-undecanethiol solution in ethanol/water (80/20) was added such that the solution was allowed to come into contact with the metal film, so that surface treatment was carried out at 25° C. for 18 hours. Thereafter, it was washed with ethanol 5 times, then with a mixed solvent of ethanol/water once, and then with water 5 times.

Thereafter, the surface coated with 11-hydroxy-1-undecanethiol was allowed to come into contact with a solution containing 10 weight % epichlorohydrin (solvent: 1:1 mixed solution of 0.4 M sodium hydroxide and diethylene glycol dimethyl ether), and the reaction was proceeded in a shaking incubator at 25° C. for 4 hours. The surface was then washed with ethanol twice, and then with water 5 times.

Thereafter, 4.5 ml of 1 M sodium hydroxide was added to 40.5 ml of an aqueous solution containing 25 weight % dextran (T500, Pharmacia), and the obtained solution was allowed to come into contact with the surface treated with epichlorohydrin. Thereafter, the surface was incubated in a shaking incubator at 25° C. for 20 hours. The surface was washed with water at 50° C. 10 times.

Subsequently, a mixture obtained by dissolving 3.5 g of bromoacetic acid in 27 g of 2 M sodium hydroxide solution was allowed to come into contact with the above dextran-treated surface, and it was then incubated in a shaking incubator at 28° C. for 16 hours. The surface was washed with water, and then, the above-described operation was repeated once. This sample is called a dextran surface chip.

(2) Production of Polymethyl Methacrylate (PMMA) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of a methyl ethyl ketone solution containing 1 mg/ml polymethyl methacrylate was added thereto such that it was allowed to come into contact with the metal film, and it was then left at rest at 25° C. for 15 minutes.

(3) Production of Measurement Chip Comprising Polymethyl Methacrylate Film Treated with NaOH

A 1N NaOH aqueous solution was added to the sample obtained in (2) above such that it was allowed to come into contact with the PMMA film. The sample was left at rest at 60° C. for 5 hours, and it was then washed with water 3 times. This sample is called a PMMA/NaOH treated chip.

(4) Production of Polystyrene (PS) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of a methyl ethyl ketone solution containing 1 mg/ml polystyrene was added thereto such that it was allowed to come into contact with the metal film, and it was then left at rest at 5° C. for 15 minutes.

(5) Production of Measurement Chip Comprising Polystyrene Film Treated with Ozone

The sample obtained in (4) above was treated with a Model-208 UV-ozone cleaning system (TECHNOVISION INC.) for 30 minutes. This sample is called a PS/ozone treated film.

(6) Production of Gelatin (Gel) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of an aqueous solution containing a mixture of 0.1 weight % gelatin and a compound A with a percentage by weight described in Table 1 or aluminum a sulfate was added thereto such that it was allowed to come into contact with the metal film. It was then left at rest at 25° C. for 15 minutes. This sample is called a gelatin film chip.

(7) Production of Polyvinyl Alcohol (PVA) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of a mixed aqueous solution of 0.1 weight % polyvinyl alcohol (MP103 manufactured by Kuraray Co., Ltd.) and Sumitex Resin M-3 (80% aqueous solution, manufactured by Sumitomo Chemical Co., Ltd.) with percentage by weight described in Table 1 was added thereto such that it was allowed to come into contact with the metal film. It was then left at rest at 25° C. for 15 minutes. This sample is called a PVA film chip.
CH₂═CHSO₂—CH₂—CONH—CH₂CH₂—NHCO—CH₂SO₂—CH═CH₂ Compound A

### Example E-2

### Evaluation of Performance of Measurement Chip

(1) Evaluation of Baseline Stability During Measurement

In particular, when binding of a low molecular weight test compound is detected, if the baseline is unstable during measurement, it becomes extremely difficult to detect the binding.

With regard to the degree of swelling of the film of each chip in water, the film was measured both in a dry state and in a state where it was swollen with pure water at 25° C., using SPA400 SPM manufactured by SII in AFM mode under a 25° C. environment. The swelling degree (ratio) of each film is shown in Table 8.

Baseline stability was evaluated by the following method. First, a measurement chip was placed on the surface plasmon resonance measurement device of the present invention, and water was added thereto. After it was left for 30 minutes, water was removed therefrom, and an HBS-N buffer (0.01 mol/l HEPES (pH 7.4); 0.15 mol/l NaCl) was added thereto. It was left at rest for 30 minutes, and the amount of change of resonance signal (RU value) during that time was recorded. The amount of change of resonance signal (RU value) from 5 to 15 minutes (A5-15) after the substitution, and the amount of change of resonance signal from 20 to 30 minutes (A20-30) after the substitution, were measured. Each of these amounts of change of resonance signal (RU values) is preferably 10 RU or lower, and more preferably 5 RU or lower. Moreover, the difference between A5-15 and A20-30 is preferably 5 RU or lower.

(4) Results

Table 8 shows the measurement results of baseline stability during measurement.

TABLE 8

| | | | hardening | | | | | |
|---|---|---|---|---|---|---|---|---|
| Sample | surface | monomer | agent type, | (weight | rate of | baseline stability | | |
| no. | Treatment | solubility | amount | %) | swelling | Δ5-15 | Δ20-30 | remarks |
| 1-1 | dextran | — | — | | 30 | −53 | −29 | Comparative |
| 1-2 | PMMA | 1.35 weight % | — | | 1 | −3 | −2 | Invention |
| 1-3 | PMMA/NaOH | 1.35 weight % | — | | 1.05 | −5 | −3 | Invention |
| 1-4 | PS | 0.03 weight % | — | | 1 | −2 | −1 | Invention |
| 1-5 | PS/ozone | 0.03 weight % | — | | 1.1 | −7 | −4 | Invention |
| 1-6-1 | gelatin-1 | — | — | | 20 | −48 | −21 | Comparative |
| 1-6-2 | gelatin-2 | — | Compound A | 0.0001 | 10 | −34 | −12 | Comparative |
| 1-6-3 | gelatin-3 | — | Compound A | 0.001 | 5 | −20 | −9 | Invention |
| 1-6-4 | gelatin-4 | — | Compound A | 0.01 | 2 | −9 | −6 | Invention |
| 1-6-5 | gelatin-5 | — | Al₂(SO₄)₃ | 0.0001 | 8 | −25 | −16 | Comparative |
| 1-6-6 | gelatin-6 | — | Al₂(SO₄)₃ | 0.001 | 3 | −10 | −9 | Invention |
| 1-6-7 | gelatin-7 | — | Al₂(SO₄)₃ | 0.01 | 1.2 | −5 | −3 | Invention |
| 1-7-1 | PVA-1 | ∞ | — | | 25 | −19 | −12 | Comparative |
| 1-7-2 | PVA-2 | ∞ | M-3 | 0.001 | 12 | −12 | −8 | Comparative |
| 1-7-3 | PVA-3 | ∞ | M-3 | 0.01 | 4 | −5 | −4 | Invention |

From the results shown in Table 8, it has been found that when the biosensor of the present invention comprising a substrate coated with a film whose swelling degree in pure water at 25° C. is between 1 and 5 with respect to the film thickness in a dry state is used, baseline can be stabilized during measurement.

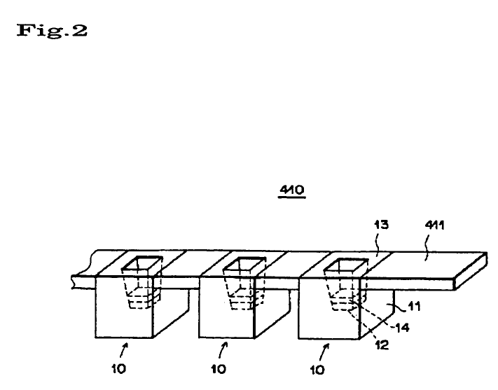

The following experiment was carried out using a device shown in FIG. 22 of Japanese Patent Laid-Open No. 2001-330560 (hereinafter referred to as the surface plasmon resonance measurement device of the present invention) (shown in FIG. 1 of the present specification) and a dielectric block shown in FIG. 23 of Japanese Patent Laid-Open No. 2001-330560 (hereinafter referred to as the dielectric block of the present invention) (shown in FIG. 2 of the present specification).

In the surface plasmon resonance measurement device shown in FIG. 1, a slide block 401 is used as a supporting medium for supporting measurement units, which is joined to two guide rods 400, 400 placed in parallel with each other while flexibly sliding in contact, and which also flexibly moves linearly along the two rods in the direction of an arrow Y in the figure. The slide block 401 is screwed together with a precision screw 402 placed in parallel with the above guide rods 400, 400, and the precision screw 402 is reciprocally rotated by a pulse motor 403 which constitutes a supporting medium-driving means together with the precision screw 402.

It is to be noted that the movement of the pulse motor 403 is controlled by a motor controller 404. This is to say, an output signal S 40 of a linear encoder (not shown in the figure), which is incorporated into the slide block 401 and detects the position of the slide block 401 in the longitudinal direction of the guide rods 400, 400, is inputted into the motor controller 404. The motor controller 404 controls the movement of the pulse motor 403 based on the signal S 40.

Moreover, below the guide rods 400, 400, there are established a laser light source 31 and a condenser 32 such that they sandwich from both sides the slide block 401 moving along the guide rods, and a photodetector 40. The condenser 32 condenses a light beam 30. In addition, the photodetector 40 is placed thereon.

In an embodiment of the present invention, a stick-form unit connected body 410 obtained by connecting and fixing eight measurement units 10 is used as an example, and the measurement units 10 are mounted on the slide block 401 in a state in which eight units are arranged in a line.

FIG. 2 shows the structure of the unit connected body 410 in detail. As shown in the figure, the unit connected body 410 is obtained by connecting the eight measurement units 10 by a connecting member 411.

This measurement unit 10 is obtained by molding a dielectric block 11 and a sample-retaining frame 13 into one piece, for example, using transparent resin or the like. The measurement unit constitutes a measurement chip that is exchangeable with respect to a turntable. In order to make the measurement chip exchangeable, for example, the measurement unit 10 may be fitted into a through-hole that is formed in the turntable. In the present example, a sensing substance 14 is immobilized on a metal film 12.

### Example F-1

### Production of Measurement Chip

(1) Comparative Example: Production of Measurement Chip Coated With SAM Compound (7-carboxy-1-heptanethiol) (SAM: Self-Assembled Membrane)

The dielectric block of the present invention, onto which gold of a thickness of 50 nm had been evaporated as a metal film, was treated with a Model-208 UV-ozone cleaning system (TECHNOVISION INC.) for 30 minutes. Thereafter, an ethanol solution containing 1 mM 7-carboxy-1-heptanethiol (Dojin Chemicals) was added thereto such that the solution was allowed to come into contact with the metal film, followed by surface treatment at 25° C. for 18 hours. Thereafter, it was washed with ethanol 5 times, then with a mixed solvent of ethanol/water once, and then with water 5 times. This sample is called an SAM treated chip.

(2) Production of Polymethyl Methacrylate (PMMA) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of a methyl ethyl ketone solution containing 0.5 mg/ml polymethyl methacrylate was added thereto such that the solution was allowed to come into contact with the metal film. Thereafter, it was left at rest at 5° C. for 15 minutes.

(3) Production of Measurement Chip Comprising Polymethyl Methacrylate Treated with NaOH

1N NaOH aqueous solution was added to the sample obtained in (2) above such that the solution was allowed to come into contact with the PMMA film, and it was then left at rest at 60° C. for 5 hours, followed by washing with water 3 times. This sample is called a PMMA NaOH treated chip.

(4) Production of Polystyrene (PS) Film

The dielectric block of the present invention, onto which gold of a thickness of 50 nm had been evaporated as a metal film, was treated with a Model-208 UV-ozone cleaning system (TECHNOVISION INC.) for 30 minutes. Thereafter, 5 μl of a methyl ethyl ketone solution containing 0.5 mg/ml polystyrene was added thereto such that the solution was allowed to come into contact with the metal film. Thereafter, it was left at rest at 5° C. for 15 minutes.

(5) Production of Measurement Chip Comprising Polystyrene Film Treated with Ozone

The sample obtained in (2) above was treated with a Model-208 UV-ozone cleaning system (TECHNOVISION INC.) for 30 minutes. This sample is called a PS/ozone treated chip.

### Example F-2

### Evaluation of Performance of Measurement Chip

(1) Measurement of Nonspecific Adsorption of Proteins

Since nonspecific adsorption of proteins on the surface of a measurement chip causes noise, such adsorption is preferably as low as possible. Nonspecific adsorption of BSA (manufactured by Sigma) and avidin (manufactured by Nacalai Tesque) was evaluated by the following method.

A mixed solution of 1-ethyl-2,3-dimethylaminopropylcarbodiimide (400 mM) and N-hydroxysuccinimide (100 mM) was added to each of the SAM treated chip (produced by the method described in Example F-1 (1)), the PMMA/NaOH treated chip (produced by the method described in Example F-1 (3)), and the PS/ozone treated chip (produced by the method described in Example F-1 (5)), and each chip was then left at rest for 20 minutes. After each measurement chip had been washed with water, an ethanolamine/HCl solution (1 M, pH 8.5) was added thereto, and it was left at rest for 20 minutes. Thereafter, the chip was washed with an HBS-EP buffer (manufactured by Biacore K.K., pH 7.4).

The HBS-EP buffer used above consisted of 0.01 mol/l (pH 7.4) HEPES (N-2-hydroxyethylpiperazine-N′-2-ethanesulfonic acid), 0.15 mol/l NaCl, 0.003 mol/l EDTA, and 0.005 weight % surfactant P20.

Subsequently, each of these measurement chips was placed in the surface plasmon resonance measurement device of the present invention. A BSA solution (2 mg/ml, HBS-EP buffer) or avidin solution (2 mg/ml, HBS-EP buffer) was added thereto, and the resultant has been left at rest for 10 minutes. Thereafter, it was washed with an HBS-EP buffer, and the amount of change of resonance signal (RU value) measured at 3 minutes later was defined as a nonspecifically adsorbed amount of each protein.

(2) Measurement of Interaction Between Protein and Test Compound

Neutral avidin (manufactured by PIERCE) was immobilized to each measurement chip, and interaction with D-biotin (manufactured by Nacalai Tesque) was measured by the method described below.

A mixed solution of 1-ethyl-2,3-dimethylaminopropylcarbodiimide (400 mM) and N-hydroxysuccinimide (100 mM) was added to each of the SAM treated chip (produced by the method described in Example F-1(1)), the PMMA/NaOH treated chip (produced by the method described in Example F-1 (3)), and the PS/ozone treated chip (produced by the method described in Example F-1 (5)), and each chip was then left at rest for 20 minutes. Thereafter, each measurement chip was washed with an HBS-N buffer (manufactured by Biacore K.K., pH 7.4). Subsequently, a neutral avidin solution (100 μg/ml, HBS-N buffer) was added thereto. After it had been left at rest for 30 minutes, it was washed with an HBS-N buffer. By this operation, neutral avidin was immobilized on the surface of each measurement chip by covalent binding. The amount of change of resonance signal (RU value) obtained before the addition of neutral avidin and after washing was defined as the immobilized amount of neutral avidin (RU value).

The HBS-N buffer used above consisted of 0.01 moll (pH 7.4) HEPES (N-2-hydroxyethylpiperazine-N′-2-ethanesulfonic acid) and 0.15 mol/l NaCl.

Thereafter, an ethanolamine/HCl solution (1 M, pH 8.5) was added to each measurement chip and then washed with an HBS-N buffer, so that COOH groups remaining without reacting with neutral avidin were blocked.

Subsequently, each measurement chip was placed in the surface plasmon resonance measurement device of the present invention. D-biotin (0.5 μg/ml, HBS-N buffer) was added to each measurement chip, and the resultant was left at rest for 10 minutes. Thereafter, it was washed with an HBS-EP buffer. The amount of change of resonance signal (RU value) obtained before the addition of D-biotin and after washing was defined as the amount of D-biotin binding to neutral avidin.

(3) Evaluation of Baseline Stability During Measurement

In particular, when binding of a low molecular weight test compound is detected, if the baseline is unstable during measurement, it becomes extremely difficult to detect the binding.

Baseline stability was evaluated by the following method. First, neutral avidin was immobilized to each measurement chip by the same method as described in (2) above. Then, the measurement chip was placed on the surface plasmon resonance measurement device of the present invention, and an HBS-N buffer was added thereto. After it had been left at rest for 30 minutes, the amount of change of resonance signal (RU value) during that time was recorded. The amount of change of resonance signal (RU value) is preferably 10 RU or lower, and more preferably 5 RU or lower.

(4) Results

Table 9 shows the measurement results regarding the nonspecific adsorption of a protein, Table 10 shows the measurement results regarding the interaction between a protein and a test compound, and Table 11 shows baseline stability during measurement.

TABLE 9

| | | nonspecific | | |
|---|---|---|---|---|
| | | adsorption | | |
| Sample | | (RU value) | | |
| No. | Surface treatment | BSA | avidin | Remarks |
| 1-1 | SAM treated membrane | 632 | 1350 | Comparative |
| 1-2 | PMMA/NaOH treated | 7 | 18 | Invention |
| | membrane | | | |
| 1-3 | PS/ozone treated membrane | 16 | 35 | Invention |

TABLE 10

| | | Binding | Binding | |
|---|---|---|---|---|
| | | amount of | amount of | |
| Sample | Surface | neutral avidin | D-biotin | |
| No. | treatment | (RU value) | (RU value) | Remarks |
| 2-1 | SAM treated | 1350 | 26 | Comparative |
| | membrane | | | |
| 2-2 | PMMA/NaOH | 1530 | 28 | Invention |
| | treated membrane | | | |
| 2-3 | PS/ozone treated | 1490 | 27 | Invention |
| | membrane | | | |

TABLE 11

| Sample | Surface | Baseline stability | |
|---|---|---|---|
| No. | treatment | (Δ RU) | Remarks |
| 1-1 | SAM treated membrane | −23 | Comparative |
| 1-2 | PMMA/NaOH treated | −3 | Invention |
| | membrane | | |
| 1-3 | PS/ozone treated membrane | −6 | Invention |

From the results shown in Table 9, it has been found that the present invention provides a surface causing an extremely small degree of nonspecific adsorption of proteins. From the results shown in Table 10, it has been found that the present invention enables immobilization of proteins and detection of a test compound as in the conventional method. From the results shown in Table 11, it has been found that the present invention enables the stabilization of the baseline during measurement. This is to say, the present invention provides a measurement chip used for a surface plasmon resonance measurement device, which represses the nonspecific adsorption of proteins and provides excellent baseline stability during measurement.

### INDUSTRIAL APPLICABILITY

According to the present invention, it becomes possible to provide a detection surface used for a biosensor which represses the nonspecific adsorption of proteins. Also, according to the present invention, it becomes possible to provide a detection surface used for a biosensor which improves baseline stability during measurement.

1. A biosensor comprising a substrate coated with a hydrophobic polymer.

2. The biosensor according to claim 1, which comprises a metal surface or metal film coated with a hydrophobic polymer.

3. The biosensor according claim 2, wherein the metal surface or metal film comprises a free-electron metal selected from a group consisting of gold, silver, copper, platinum and aluminum.

4. The biosensor according to claim 1, wherein the coating thickness of the hydrophobic polymer is between 1 angstrom and 5,000 angstroms.

5. The biosensor according to claim 1, wherein the coating thickness of the hydrophobic polymer is between 10 angstroms and 2,000 angstroms.

6. A biosensor comprising a substrate coated with a film whose swelling degree in pure water at 25° C. is between 1 and 5 with respect to the film thickness in a dry state.

7. The biosensor according to claim 6, wherein the film whose swelling degree in pure water at 25° C. is between 1 and 5 with respect to the film thickness in a dry state is an organic substance.

8. The biosensor according to claim 6, wherein the film whose swelling degree in pure water at 25° C. is between 1 and 5 with respect to the film thickness in a dry state comprises a high polymer comprising 50% by weight or more of monomers having a solubility in water of 20% by weight or less.

9. The biosensor according to claim 6, wherein the film whose swelling degree in pure water at 25° C. is between 1 and 5 with respect to the film thickness in a dry state comprises a hardening agent.

10. The biosensor according to claim 6, which comprises a metal surface or metal film coated with a film whose swelling degree in pure water at 25° C. is between 1 and 5 with respect to the film thickness in a dry state.

11. The biosensor according claim 6, wherein the metal surface or metal film comprises a free-electron metal selected from a group consisting of gold, silver, copper, platinum and aluminum.

12. The biosensor according to claim 1, which has a functional group capable of immobilizing a physiologically active substance on the outermost surface of the substrate.

13. The biosensor according to claim 12, wherein the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group.

14. The biosensor according to claim 12, which comprises a substrate coated with a hydrophobic polymer, and wherein a functional group capable of immobilizing a physiologically active substance by covalent bond is introduced in a hydrophobic polymer by chemical treatment of the surface of said substrate.

15. The biosensor according to claim 1, which comprises a linker for immobilizing a physiologically active substance on a surface of the biosensor.

16. The biosensor according to claim 15, wherein the linker is a linker for immobilizing a physiologically active substance on a surface of the biosensor by chemical bonding.

17. The biosensor according to claim 15, wherein the linker is a linker for immobilizing a physiologically active substance on a surface of the biosensor by covalent bonding.

18. The biosensor according to claim 15, wherein the linker is a compound represented by the formula (1)
X-L-Y formula (1)
wherein X represents a group capable of reacting with a functional group of a hydrophobic polymer, L represents a bivalent linking group, and Y represents a group capable of immobilizing a physiologically active substance.

19. The biosensor according to claim 18, wherein the total number of atoms of L of the formula (1) is 2 to 1000.

20. The biosensor according to claim 1, which is used in non-electrochemical detection.

21. The biosensor according to claim 1, which is used in surface plasmon resonance analysis.

22. A method for producing the biosensor according to claim 1, which comprises a step of coating a substrate with a hydrophobic polymer.

23. The method for producing the biosensor according to claim 22, which further comprises a step of performing chemical treatment of a surface of the substrate.

24. The method for producing the biosensor according to claim 22, which further comprises a step of reacting the substrate with a hydrophobic polymer with a linker.

25. The biosensor according to claim 1, wherein a physiologically active substance is bound to the surface by covalent bonding.

26. A method for immobilizing a physiologically active substance to the biosensor according to claim 1, which comprises a step of making said biosensor come into contact with said physiologically active substance, so that said physiologically active substance is bound to the surface of said biosensor by covalent bonding.

27. A method for detecting or measuring a substance interacting with a physiologically active substance, which comprises a step of making the biosensor according to claim 1, to the surface of which said physiologically active substance is bound by covalent bonding, come into contact with a test substance.

28. The method according to claim 27, wherein a substance interacting with the physiologically active substance is detected or measured by a non-electrochemical method.

29. The method according to claim 27, wherein a substance interacting with the physiologically active substance is detected or measured by surface plasmon resonance analysis.

30. A method for detecting or measuring a substance interacting with a physiologically active substance which is bound to the surface of a biosensor comprising a substrate coated with a hydrophobic polymer, wherein the above detection or measurement is carried out in the presence of a surfactant.

31. The method according to claim 30 wherein the surfactant is a nonionic surfactant.

32. The method according to claim 30 wherein a solution containing at least a test substance and a surfactant is allowed to come into contact with a biosensor comprising a substrate coated with hydrophobic polymer, on the surface of which a physiologically active substance is bound by covalent bonding.

33. The method according to claim 32 wherein the concentration of a surfactant contained in the solution containing the test substance and the surfactant is between 0.0001% by weight and 1% by weight.

34. A measurement chip used for a surface plasmon resonance measurement device comprising: a dielectric block; a metal film formed on a face of the dielectric block; a light source for generating a light beam; an optical system for allowing said light beam to enter said dielectric block such that total reflection conditions can be obtained at the interface between said dielectric block and said metal film and that components at various incident angles can be contained; and a light-detecting means for detecting the state of surface plasmon resonance by measuring the intensity of the light beam totally reflected at said interface,
said measurement chip being comprised of said dielectric block and said metal film, wherein said dielectric block is formed as one block comprising the entirety of the entrance face and exit face of said light beam and a face on which said metal film is formed, said metal film is integrated with the dielectric block, and said metal film is coated with a hydrophobic polymer.

35. The measurement chip according to claim 34, which has a functional group capable of immobilizing a physiologically active substance on the surface of the metal film coated with a hydrophobic polymer.

36. The measurement chip according to claim 35, wherein the functional group capable of immobilizing a physiologically active substance is —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group.

37. The measurement chip according to claim 34, wherein the physiologically active substance is bound to the surface by covalent bonding.

38. A method for immobilizing a physiologically active substance to a measurement chip, which comprises a step of allowing the measurement chip according to claim 34 to come into contact with the physiologically active substance, so as to bind the physiologically active substance to the surface of the measurement chip by covalent bonding.

39. A method for detecting or measuring a substance interacting with a physiologically active substance, which comprises a step of allowing the measurement chip according to claim 34, on the surface of which the physiologically active substance is bound by covalent bonding, to come into contact with a test substance.

40. A surface plasmon resonance measurement device having the measurement chip according to claim 34.
